(19) United States
(12) Patent Application Publication
(10) Pub. No.: US 2026/0108748 A1
(43) Pub. Date: Apr. 23, 2026

(54) AUTOMATED EXTERNAL DEFIBRILLATOR

(71) Applicant: Nihon Kohden Corporation, Tokyo (JP)

(72) Inventors: Yutaka Tsujimoto, Saitama (JP); Ryosuke Kuno, Saitama (JP); Yuji Yamada, Saitama (JP); Hideto Sakakibara, Saitama (JP); Fumihito Iwai, Saitama (JP)

(21) Appl. No.: 19/362,245

(22) Filed: Oct. 17, 2025

(30) Foreign Application Priority Data
Oct. 18, 2024 (JP) 2024-183939
Nov. 15, 2024 (JP) 2024-199591

(51) Int. Cl. A61N 1/39; A61N 1/37

(52) U.S. Cl. CPC A61N 1/3993; A61N 1/3702; A61N 1/39044; A61N 1/3925; A61N 1/3968; A61N 1/3975

(57) ABSTRACT

An automated external defibrillator for performing electrocardiogram analysis and discharge processing on a subject. The automated external defibrillator includes a detection unit configured to detect a body motion of the subject, in a cardiopulmonary resuscitation period after the electrocardiogram analysis and the discharge processing, and an output controller configured to output, to a rescuer, an instruction regarding chest compression on the subject, based on presence or absence of the body motion of the subject, in the cardiopulmonary resuscitation period. The output controller is configured to output a continuation instruction for instructing continuation of chest compression, and in a case where a first time has elapsed from the output of a previous continuation instruction, output the continuation instruction again.

### CROSS-REFERENCE TO RELATED APPLICATIONS

This application is based on and claims priority under 35 USC 119 from Japanese Patent Application No. 2024-183939 filed on Oct. 18, 2024 and Japanese Patent Application No. 2024-199591 filed on Nov. 15, 2024, the entire content of which is incorporated herein by reference.

### TECHNICAL FIELD

The presently disclosed subject matter relates to an automated external defibrillator.

“JRC Resuscitation Guidelines 2020,” published by Igaku Shoin on Jun. 15, 2021 (hereinafter “JRC Resuscitation Guidelines 2020”) defines that chest compression and respirator for a subject are continued during a cardiopulmonary resuscitation (CPR) period after electrocardiogram analysis and electric shock by an automated external defibrillator (AED).

### BACKGROUND ART

An automated external defibrillator (hereinafter, also referred to as “AED”) that periodically performs voice notification of a continuation instruction of chest compression in a cardiopulmonary resuscitation period (hereinafter, also referred to as “CPR period”) based on the number of times of chest compression and respirator determined in JRC Resuscitation Guidelines 2020″ is known. However, since the continuation instruction of chest compression by the automated external defibrillator is periodically notified regardless of whether the rescuer performs chest compression, for example, when the rescuer fails to hear the continuation instruction, it may take a long time to grasp what kind of treatment is to be performed next.

An AED is known in which the continuation instruction of chest compression is notified by sound and blinking of a lamp during a CPR period. However, since blinking of the lamp is continuously performed in the same cycle regardless of whether the rescuer performs chest compression, there is room for more effective instruction to the rescuer.

### SUMMARY OF INVENTION

Aspect of non-limiting embodiments of the present disclosure relates to provide an automated external defibrillator capable of more appropriately supporting movement of a rescuer in a cardiopulmonary resuscitation period and to provide an automated external defibrillator capable of giving a more effective instruction to a rescuer.

Aspects of certain non-limiting embodiments of the present disclosure address the features discussed above and/or other features not described above. However, aspects of the non-limiting embodiments are not required to address the above features, and aspects of the non-limiting embodiments of the present disclosure may not address features described above.

According to an aspect of the present disclosure, there is provided an automated external defibrillator for performing electrocardiogram analysis and discharge processing on a subject, the automated external defibrillator including:
a detection unit configured to detect a body motion of the subject, in a cardiopulmonary resuscitation period after the electrocardiogram analysis and the discharge processing; and
an output controller configured to output, to a rescuer, an instruction regarding chest compression on the subject, based on presence or absence of the body motion of the subject, in the cardiopulmonary resuscitation period,
in which the output controller is configured to:
   output a continuation instruction for instructing continuation of chest compression;
   in a case where a first time has elapsed from the output of a previous continuation instruction, output the continuation instruction again;
   in a case where no body motion of the subject is continuously detected for a second time shorter than the first time or for longer than the second time, output a start instruction for instructing a start of the chest compression in priority to the continuation instruction; and
   in a case where the second time has elapsed from the output of a previous start instruction, output the start instruction again.
      According to an aspect of the present disclosure, there is provided an automated external defibrillator for performing electrocardiogram analysis and discharge processing on a subject, the automated external defibrillator including:
a detection unit configured to detect whether a rescuer is in contact with the subject; and
a display controller configured to change a blinking cycle or a lighting color of a display unit provided in the automated external defibrillator, according to a detection result obtained by the detection unit.

### BRIEF DESCRIPTION OF DRAWINGS

Exemplary embodiment(s) of the present invention will be described in detail based on the following figures, wherein:

FIG. 1 is an external view of an AED according to a first embodiment of the presently disclosed subject matter;

FIG. 2 is a block diagram illustrating the configuration of the AED illustrated in FIG. 1;

FIG. 3 is a diagram illustrating the output of guidance in a CPR period by the AED according to a comparative example;

FIG. 4 is a flowchart illustrating an example of a procedure of outputting guidance in a CPR period by the AED according to the first embodiment of the presently disclosed subject matter;

FIG. 5 is a diagram illustrating an example (a specific example 1) of outputting guidance in a CPR period by the AED according to the presently disclosed subject matter;

FIG. 6 is a diagram illustrating an example (a specific example 2) of outputting guidance in a CPR period by the AED according to the presently disclosed subject matter;

FIG. 7 is a diagram illustrating an example (a specific example 3) of outputting guidance in a CPR period by the AED according to the presently disclosed subject matter;

FIG. 8 is a diagram illustrating an example (a specific example 4) of outputting guidance in a CPR period by the AED according to the presently disclosed subject matter;

FIG. 9 is a diagram illustrating an example (a specific example 5) of outputting guidance in a CPR period by the AED according to the presently disclosed subject matter;

FIG. 10 is a diagram illustrating an example (a specific example 6) of outputting guidance in a CPR period by the AED according to the presently disclosed subject matter;

FIG. 11 is a diagram illustrating an example (a specific example 7) of outputting guidance in a CPR period by the AED according to the presently disclosed subject matter;

FIG. 12 is an external view of an AED according to a second embodiment of the presently disclosed subject matter;

FIG. 13 is a block diagram illustrating the configuration of the AED illustrated in FIG. 12;

FIG. 14 is a flowchart illustrating an outline of a procedure of operations of the AED illustrated in FIG. 13;

FIG. 15 is a table illustrating a specific example of lighting control of a leaving notification lamp in a leaving period by a display controller illustrated in FIG. 13; and

FIG. 16 is a table illustrating a specific example of lighting control of a chest compression notification lamp in a CPR period by the display controller illustrated in FIG. 13.

### DESCRIPTION OF EMBODIMENTS

Hereinafter, an automated external defibrillator according to the presently disclosed subject matter will be described with reference to the drawings. Members having the same reference signs as those already described in the description of the embodiments will not be described for convenience of description. For convenience of description, the dimensions of the members illustrated in the drawings may be different from actual dimensions of the members.

### First Embodiment

### Configuration of AED

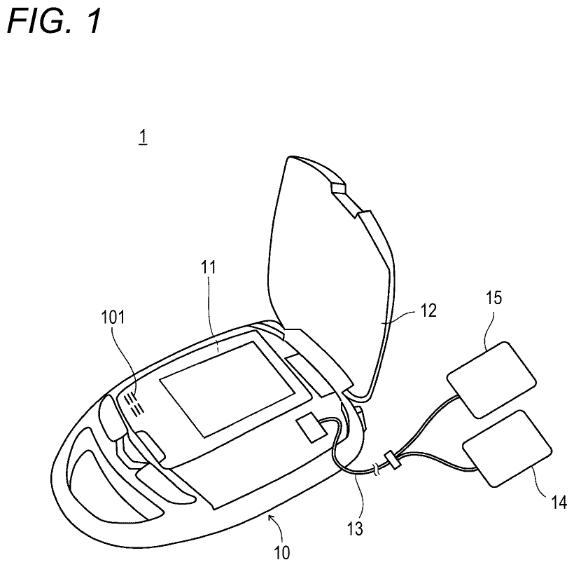

Here, an AED 1 will be described which is an example of an automated external defibrillator according to a first embodiment of the presently disclosed subject matter. FIG. 1 is an external view of the AED 1 according to the first embodiment of the presently disclosed subject matter. As illustrated in FIG. 1, the AED 1 can include an AED body 10, a battery pack 11, a lid portion 12, a cable 13, and a pair of electrode pads 14 and 15.

The battery pack 11 is configured to supply power for operating the AED body 10 to the AED body 10. For example, the battery pack 11 is detachably connected to the back side of the AED body 10.

The lid portion 12 is a structure that covers the AED body 10. When the lid portion 12 changes from the closed state to the open state, the main power supply of the AED 1 is turned on, and the AED 1 is started up. When the lid portion 12 changes from the open state to the closed state, the main power supply of the AED 1 is turned off. The AED 1 is not limited to a configuration in which the main power supply is switched ON/OFF in response to opening and closing of the lid portion 12. For example, a power button configured to switch the main power supply ON/OFF may be provided on the AED body 10.

Each of the electrode pads 14 and 15 can include a gel having viscosity. When the AED 1 is not in use, the electrode pads 14 and 15 are accommodated in a bag, in a state in which release paper is attached to each gel. The bag that accommodates the electrode pads 14 and 15 is attached to the inside of the lid portion 12, for example. A rescuer who uses the AED 1 removes the bag from the lid portion 12 and takes out the electrode pads 14 and 15 from the bag. Then, the rescuer attaches the gel portion to the skin of a subject in a state in which the electrode pads 14 and 15 are peeled off from the release paper.

The cable 13 electrically connects the AED body 10 and the electrode pads 14 and 15. In a state in which the electrode pads 14 and 15 are attached to the subject, the AED body 10 performs processing such as electrocardiogram analysis and electric shock (hereinafter also referred to as “discharge processing”) on the subject.

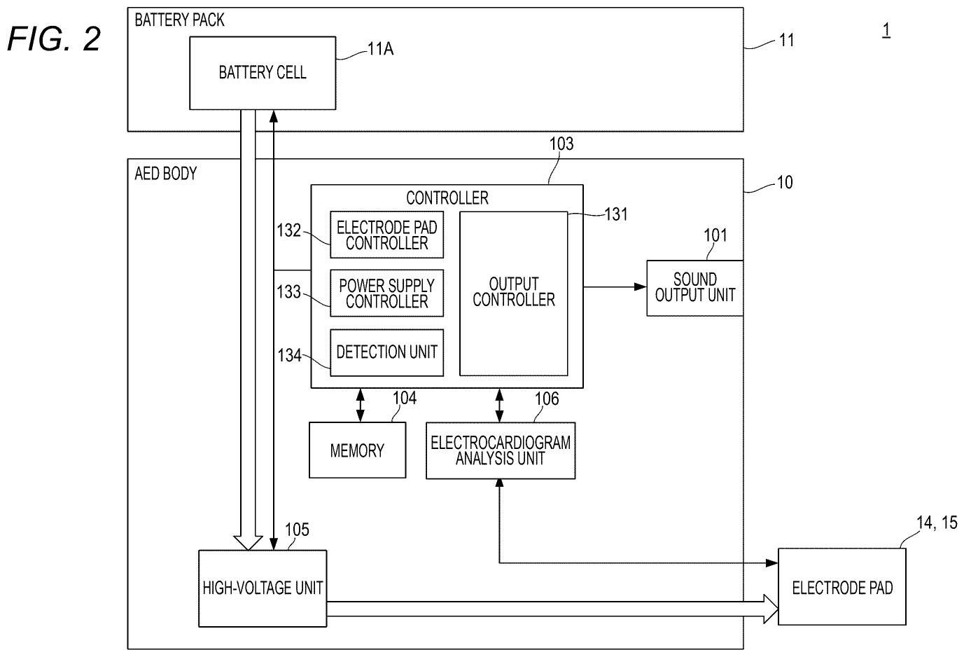

FIG. 2 is a block diagram illustrating the configuration of the AED 1 illustrated in FIG. 1. As illustrated in FIG. 2, the AED body 10 can include a sound output unit 101, a controller 103, a memory 104, a high-voltage unit 105, and an electrocardiogram analysis unit 106. The memory 104 is configured to store a program or the like for controlling various operations of the AED 1.

When the AED 1 is started up, the controller 103 reads and executes the program or the like that is stored in the memory 104, and controls the various operations of the AED 1. The controller 103 can include a central processing unit (CPU), a read only memory (ROM), a random access memory (RAM), a hard disk drive (HDD), and the like. More specifically, the controller 103 is configured to function as an output controller 131, an electrode pad controller 132, a power supply controller 133, and a detection unit 134.

The output controller 131 is configured to read sound data such as voice guidance and warning sound for the rescuer from the memory 104, and to output the read sound data to the sound output unit 101. The sound output unit 101 is, for example, a speaker. Upon receiving the sound data output from the output controller 131, the sound output unit 101 outputs voice or warning sound based on the sound data.

The electrode pad controller 132 is configured to control the electrode pads 14 and 15 and the electrocardiogram analysis unit 106 such that the electrocardiogram analysis unit 106 executes the electrocardiogram analysis. The electrocardiogram analysis unit 106 is configured to execute electrocardiogram analysis of the subject through the electrode pads 14 and 15 by being controlled by the electrode pad controller 132.

After the electrocardiogram analysis by the electrode pad controller 132, the power supply controller 133 executes control such that discharge processing on the subject is performed. Specifically, the power supply controller 133 is configured to control a battery cell 11A provided in the battery pack 11 and the internal capacitor of the high-voltage unit 105 such that the internal capacitor is charged by the battery cell 11A and discharged from the electrode pads 14 and 15.

The detection unit 134 is configured to detect the body motion of the subject. For example, the detection unit 134 is configured to obtain the impedance between the pair of electrode pads 14 and 15, and detect the body motion of the subject based on a change in the impedance. More specifically, in the cardiopulmonary resuscitation period after the electrocardiogram analysis on the subject by the electrode pad controller 132 and the discharge processing on the subject by the power supply controller 133 are performed, the detection unit 134 detects the presence or absence of the body motion of the subject at an interval of one second using, for example, the amplitude and the cycle of the impedance.

After the CPR period of approximately 2 minutes ends, the electrode pad controller 132 causes the electrocardiogram analysis unit 106 to execute the electrocardiogram analysis again. After the electrocardiogram analysis, the power supply controller 133 executes control such that the discharge processing on the subject is performed again.

### Output of Guidance in CPR Period

As described above, the output controller 131 in the AED 1 is configured to output the voice data to the sound output unit 101 to output the guidance to the rescuer. Hereinafter, the output of guidance in a CPR period by an AED according to a comparative example and the output of the guidance in the CPR period by the AED 1 according to the first embodiment of the presently disclosed subject matter will be described.

### Description of Comparative Example

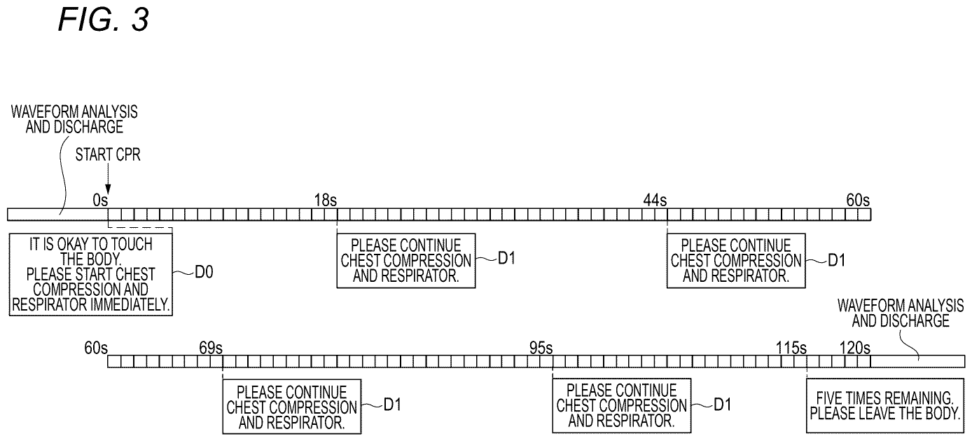

FIG. 3 is a diagram illustrating the output of the guidance in the CPR period by the AED according to the comparative example. As illustrated in FIG. 3, in the AED according to the comparative example, first, pre-start guidance D0 is output before the start of the CPR period. For example, as illustrated in FIG. 3, from approximately 7 seconds before the start timing of the CPR period, a voice message that “It is okay to touch the body. Please start chest compression and respirator immediately.”is output as the pre-start guidance D0.

When the CPR period starts, a continuation instruction D1 instructing continuation of chest compression is output every time a first time elapses. The AED according to the comparative example outputs, for example, a voice message that “Please continue chest compression and respirator” as the output of the continuation instruction D1. For example, the first time is set according to the number of times of chest compression and respirator defined in JRC Resuscitation Guidelines 2020″, and is approximately 25 seconds.

Specifically, as illustrated in FIG. 3, time measurement is started from the output timing of the pre-start guidance D0, and the first continuation instruction D1 is output 18 seconds after the start timing of the CPR period. Thereafter, the continuation instruction D1 is output 44 seconds, 69 seconds, and 95 seconds after the start timing of the CPR period.

The AED according to the comparative example outputs guidance for instructing the rescuer to move away from the subject at a timing (115 seconds after the start timing of the CPR period in the example illustrated in FIG. 3) a predetermined time before the end of the CPR period. For example, the AED outputs a voice message that “Five times remaining. Please leave the body.” as the guidance.

### Outline of Voice Notification by AED According to Presently Disclosed Subject Matter

Referring again to FIG. 2, the output controller 131 of the AED 1 according to the presently disclosed subject matter is configured to change the content of guidance for the rescuer regarding chest compression according to the presence or absence of the body motion of the subject in the CPR period.

More specifically, when the body motion of the subject is detected, the output controller 131 outputs the continuation instruction D1 every time the first time elapses, same or similarly to the AED according to the comparative example. On the other hand, when the body motion of the subject is not continuously detected for a second time or more, the output controller 131 determines that the chest compression is not performed on the subject, and outputs a start instruction D2 for instructing the start of the chest compression in priority to the continuation instruction D1.

The first time is, for example, 25 seconds, and includes a time slightly shifted from 25 seconds. The second time is shorter than the first time. The second time is, for example, 10 seconds, and includes a time slightly shifted from 10 seconds.

Specifically, when the body motion of the subject is not detected by the detection unit 134 in the last 10 seconds, the output controller 131 outputs the start instruction D2 of chest compression. For example, a voice message that “Please start chest compression immediately.” or the like is output as the output of the start instruction D2. When the situation continues in which the body motion of the subject is not detected, the output controller 131 outputs the start instruction D2 every time the second time (approximately 10 seconds) elapses from the output timing of the previous start instruction D2.

When no body motion of the subject is detected by the output timing of the first start instruction D2 in the CPR period, the output controller 131 outputs a method notification D3 for notifying the rescuer of the method of chest compression instead of the first start instruction D2. Specifically, a voice message that “Place the base of the palm on the center of the chest and place the other hand on top thereof. Please extend both elbows straight and push from directly above so as to sink by 5 centimeters or more in time with the rhythm.” or the like is output as the output of the method notification D3.

### Procedure of Operations

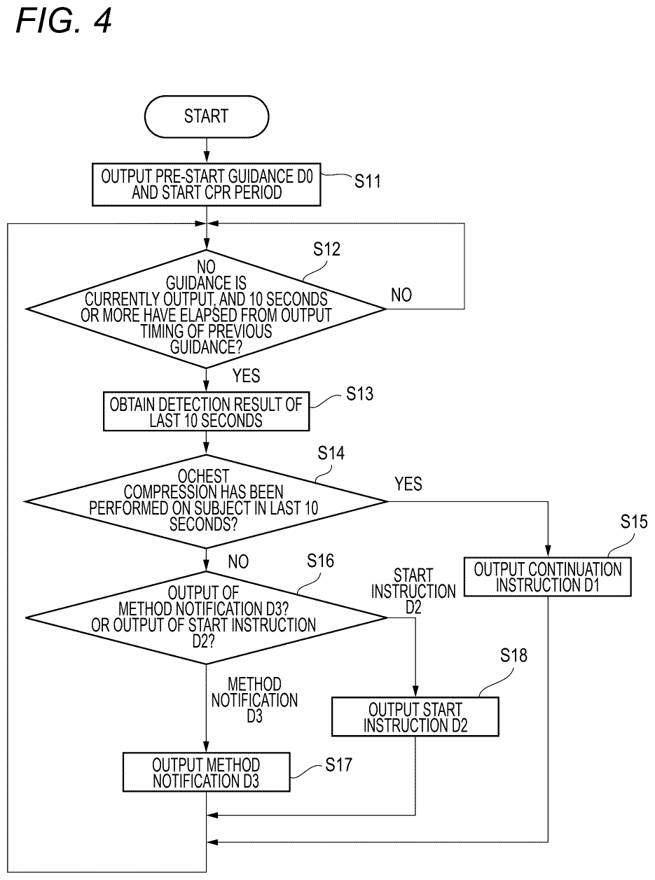

FIG. 4 is a flowchart illustrating an example of a procedure of outputting guidance in the CPR period by the AED 1 according to the first embodiment of the presently disclosed subject matter. First, the output controller 131 outputs the pre-start guidance D0 before the start of the CPR period, and then the CPR period is started (step S11). In the CPR period, the detection unit 134 detects the presence or absence of the body motion of the subject, for example, every second.

Next, in the CPR period, the output controller 131 checks whether any guidance of the pre-start guidance D0, the continuation instruction D1, the start instruction D2, and the method notification D3 is currently output, and further checks whether 10 seconds or more have elapsed from the output timing of the previous guidance (step S12).

Here, it is assumed that the guidance is currently output or 10 seconds or more have not elapsed from the output timing of the previous guidance (“NO” in step S12). In this case, the output controller 131 does not output the guidance, and executes the determination in step S12 again, for example, after a predetermined time.

On the other hand, it is assumed that the guidance is not currently output and 10 seconds or more have elapsed from the output timing of the previous guidance (“YES” in step S12). In this case, the output controller 131 obtains the detection result of the last 10 seconds by the detection unit 134 (step S13).

Next, the output controller 131 checks whether chest compression has been performed on the subject in the last 10 seconds based on the detection result obtained by the detection unit 134 (step S14). When the body motion of the subject is detected in the last 10 seconds, the output controller 131 determines that chest compression has been performed on the subject in the last 10 seconds (“YES” in step S14), and outputs the continuation instruction D1 at a predetermined timing (step S15).

On the other hand, when the body motion of the subject is not detected in the last 10 seconds, the output controller 131 determines that the chest compression is not performed on the subject in the last 10 seconds (“NO” in step S14). Then, the output controller 131 determines either the method notification D3 or the start instruction D2 as the guidance to be output (step S16).

For example, when no body motion is detected after the start of the CPR period and the first start instruction D2 is not output, the output controller 131 determines the method notification D3 as the guidance to be output (the “method notification D3” in step S16). Then, the output controller 131 outputs the method notification D3 (step S17).

On the other hand, it is assumed that there is a timing at which the body motion is detected after the start of the CPR period. In this case, the output controller 131 determines the start instruction D2 as the guidance to be output (the “start instruction D2” in step S16). Then, the output controller 131 outputs the start instruction D2 (step S18). Then, until the CPR period ends, the operations in step S12 and subsequent steps are repeated. Hereinafter, a specific example of the output of the guidance by the output controller 131 will be described.

(Specific Example 1) Case in which Body Motion is Detected in Entire CPR Period

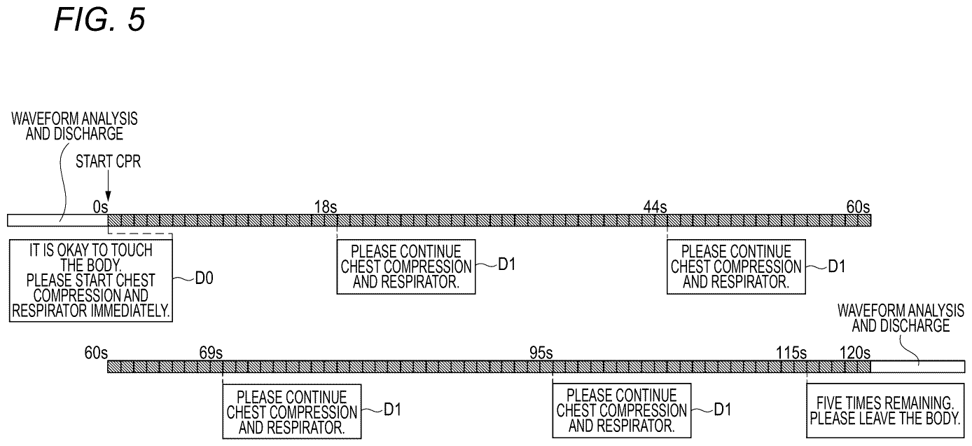

FIG. 5 is a diagram illustrating an example (a specific example 1) of outputting guidance in the CPR period by the AED 1 according to the presently disclosed subject matter. Here, it is assumed that the body motion of the subject is detected by the detection unit 134 (see FIG. 2) in the entire CPR period. In FIG. 5, a region indicating a period in which the body motion of the subject is detected is hatched.

In this case, first, in the AED 1, same or similarly to the AED according to the comparative example illustrated in FIG. 3, the pre-start guidance D0 is output approximately 7 seconds before the start timing of the CPR period. The time measurement is started from the output timing of the pre-start guidance D0, and the continuation instruction D1 is output every time approximately 25 seconds elapse. In the example illustrated in FIG. 5, the continuation instruction D1 is output 18 seconds, 44 seconds, 69 seconds, and 95 seconds after the start timing of the CPR period.

### (Specific Example 2) Case in Which no Body Motion is Detected in Entire CPR Period

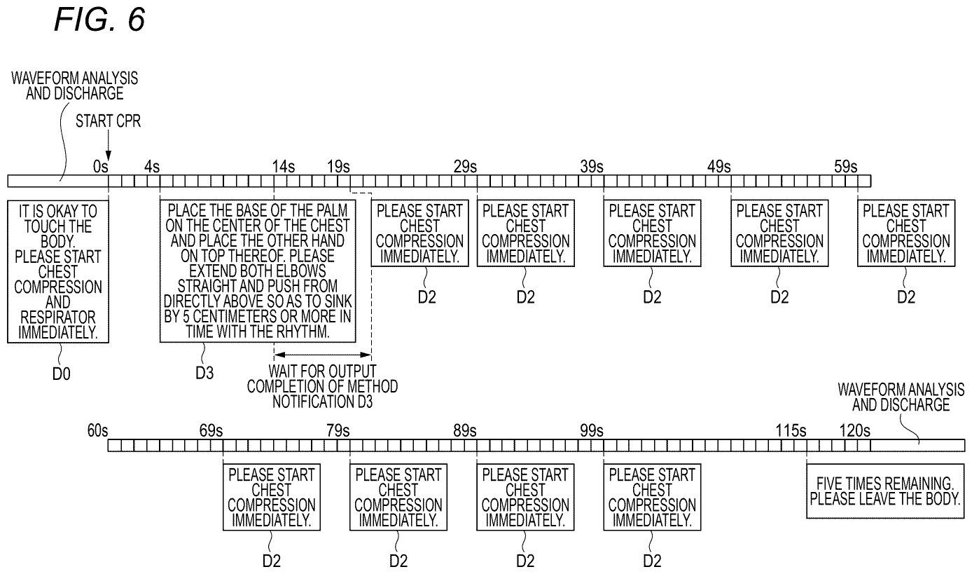

FIG. 6 is a diagram illustrating an example (a specific example 2) of outputting guidance in the CPR period by the AED 1 according to the presently disclosed subject matter. Here, it is assumed that no body motion of the subject is detected by the detection unit 134 (see FIG. 2) in the entire CPR period.

In this case, in the AED 1, the pre-start guidance D0 is output before the start of the CPR period. Then, the time measurement is started from the output timing of the pre-start guidance D0, and since no body motion of the subject is detected until approximately 10 seconds elapse, the method notification D3 of chest compression is output instead of the first start instruction D2. In the example illustrated in FIG. 6, the method notification D3 is output four seconds after the start timing of the CPR period.

Then, since no body motion of the subject is detected until approximately 10 seconds elapse from the output timing of the method notification D3, the output timing of the next start instruction D2 occurs. In the example illustrated in FIG. 6, 14 seconds after the start timing of the CPR period is the output timing of the next start instruction D2.

Here, when the output timing of the start instruction D2 is included in the output period of the method notification D3, the output controller 131 outputs the start instruction D2 after the output of the method notification D3 is completed. In the example illustrated in FIG. 6, the output timing (that is, 14 seconds after the start timing of the CPR period) of the next start instruction D2 is included in the output period of the method notification D3. Therefore, the output controller 131 outputs the next start instruction D2 after the output of the method notification D3 is completed, specifically, 19 seconds after the start timing of the CPR period.

Thereafter, the start instruction D2 is output every time approximately 10 seconds elapse. In the example illustrated in FIG. 6, the start instruction D2 is output 29 seconds, 39 seconds, 49 seconds, 59 seconds, 69 seconds, 79 seconds, 89 seconds, and 99 seconds after the start timing of the CPR period.

(Specific Example 3) Case in Which Body Motion is Detected Immediately after Start of CPR Period and then no Body Motion is Detected

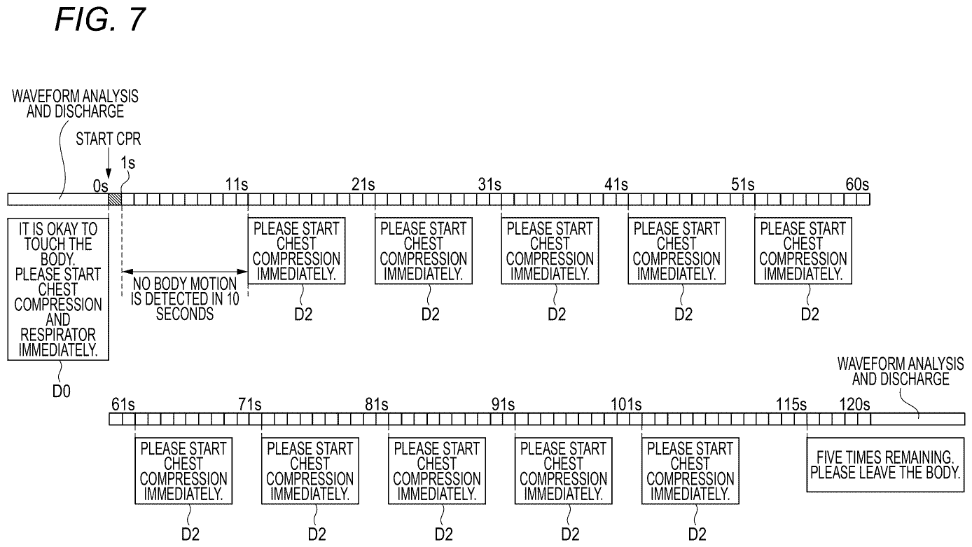

FIG. 7 is a diagram illustrating an example (a specific example 3) of outputting guidance in the CPR period by the AED 1 according to the presently disclosed subject matter. Here, it is assumed that the body motion of the subject is detected by the detection unit 134 (see FIG. 2) from the start of the CPR period to one second later, and no body motion is detected after one second. In FIG. 7, a region indicating a period in which the body motion of the subject is detected is hatched.

In this case, in the AED 1, the pre-start guidance D0 is output before the start of the CPR period, and the time measurement is started from the output timing of the pre-start guidance D0. Since the body motion of the subject is detected by the output timing of the first start instruction D2, the method notification D3 as illustrated in FIG. 6 is not output. The first start instruction D2 is output at a timing (11 seconds after the start timing of the CPR period in the example illustrated in FIG. 7) in a case where approximately 10 seconds have elapsed from a timing (1 second after the start timing of the CPR period in the example illustrated in FIG. 7) when the body motion of the subject is no longer detected.

Thereafter, since no body motion of the subject is detected, the start instruction D2 is output every time approximately 10 seconds elapse. In the example illustrated in FIG. 7, the start instruction D2 is output 21 seconds, 31 seconds, 41 seconds, 51 seconds, 61 seconds, 71 seconds, 81 seconds, 91 seconds, and 101 seconds after the start timing of the CPR period.

(Specific Example 4) Case in Which no Body Motion is Detected Until Middle of CPR Period and then Body Motion is Detected

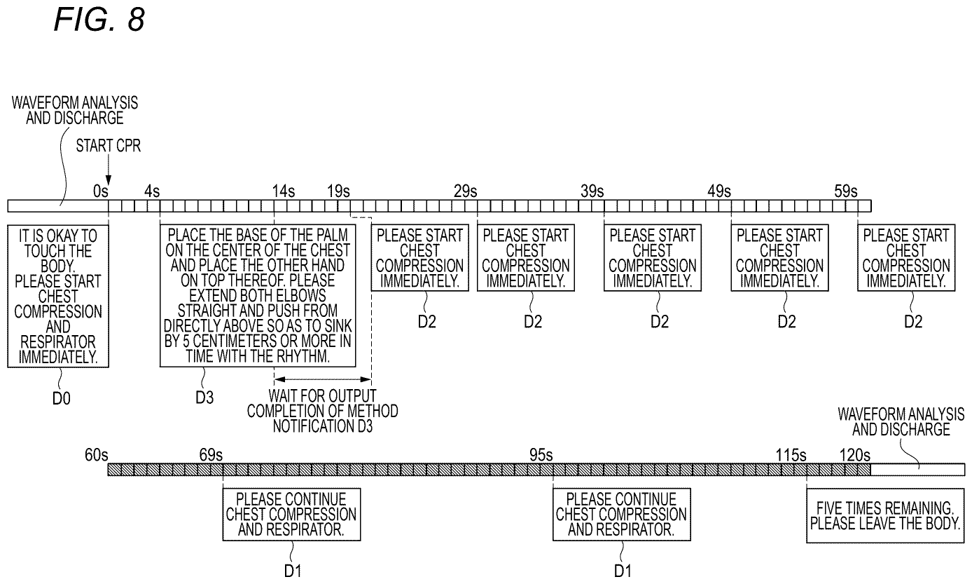

FIG. 8 is a diagram illustrating an example (a specific example 4) of outputting guidance in the CPR period by the AED 1 according to the presently disclosed subject matter. Here, it is assumed that no body motion of the subject is detected by the detection unit 134 (see FIG. 2) for 60 seconds after the start of the CPR period, and the body motion is detected after 60 seconds. In FIG. 8, a region indicating a period in which the body motion of the subject is detected is hatched.

In this case, in the AED 1, the pre-start guidance D0 is output before the start of the CPR period. Then, in the period from the start of the CPR period to 59 seconds later, the method notification D3 is output 4 seconds after the start timing of the CPR period, and the start instruction D2 is output 19 seconds later, 29 seconds later, 39 seconds later, 49 seconds later, and 59 seconds later, same or similarly to the specific example 2 illustrated in FIG. 6.

Since the body motion of the subject is detected after 60 seconds from the start timing of the CPR period, the continuation instruction D1 is output at a predetermined timing that occurs approximately every 25 seconds, same or similarly to the case in the specific example 1 illustrated in FIG. 5. In the example illustrated in FIG. 8, the continuation instruction D1 is output 69 seconds and 95 seconds after the start timing of the CPR period.

(Specific Example 5) Case in Which Start Instruction D2 is being Output at Output Timing of Continuation Instruction D1

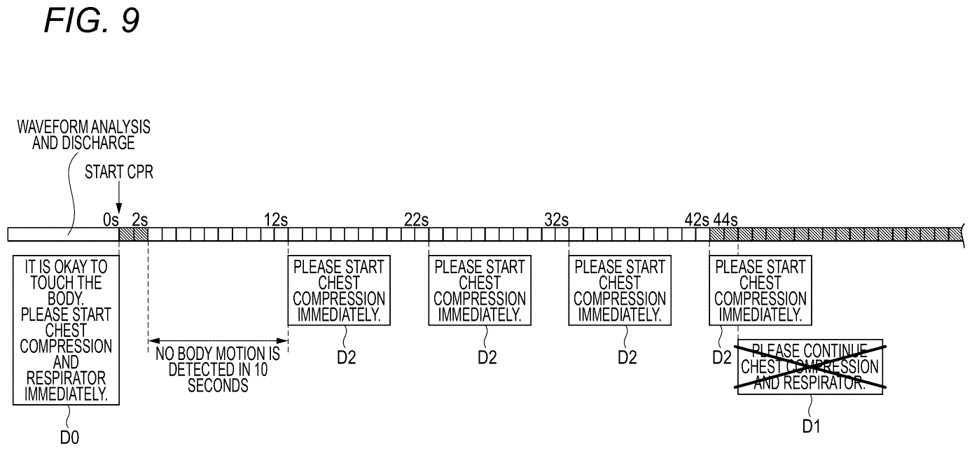

FIG. 9 is a diagram illustrating an example (a specific example 5) of outputting guidance in the CPR period by the AED 1 according to the presently disclosed subject matter. Here, it is assumed that the body motion of the subject is detected immediately after the start of the CPR period, then no body motion of the subject is detected until 42 seconds later, and the body motion of the subject is detected again after 42 seconds. In FIG. 9, a region indicating a period in which the body motion of the subject is detected is hatched.

In this case, the pre-start guidance D0 is output before the start of the CPR period, and the first start instruction D2 is output at a timing (12 seconds after the start timing of the CPR period in the example illustrated in FIG. 9) when approximately 10 seconds have elapsed from a timing (2 seconds after the start timing of the CPR period in the example illustrated in FIG. 9) when the body motion of the subject is no longer detected. Thereafter, the start instruction D2 is output every time approximately 10 seconds elapse (in the example illustrated in FIG. 9, 22 seconds, 32 seconds, and 42 seconds after the start timing of the CPR period).

Since the body motion of the subject is detected after 42 seconds from the start timing of the CPR period, the continuation instruction D1 is output at a predetermined timing that occurs approximately every 25 seconds after 42 seconds. Here, it is assumed that 44 seconds after the start timing of the CPR period is the next output timing of the continuation instruction D1. However, the output timing of the continuation instruction D1 is included in the output period of the start instruction D2.

In such a case, for example, as illustrated in FIG. 9, the output controller 131 does not output the continuation instruction D1. Accordingly, it is possible to prevent the continuation instruction D1 from being output during the output of the start instruction D2. The output controller 131 may output the continuation instruction D1 after the output of the start instruction D2 is completed.

### (Specific Example 6) Case in Which Body Motion is Detected During Output of Start Instruction D2

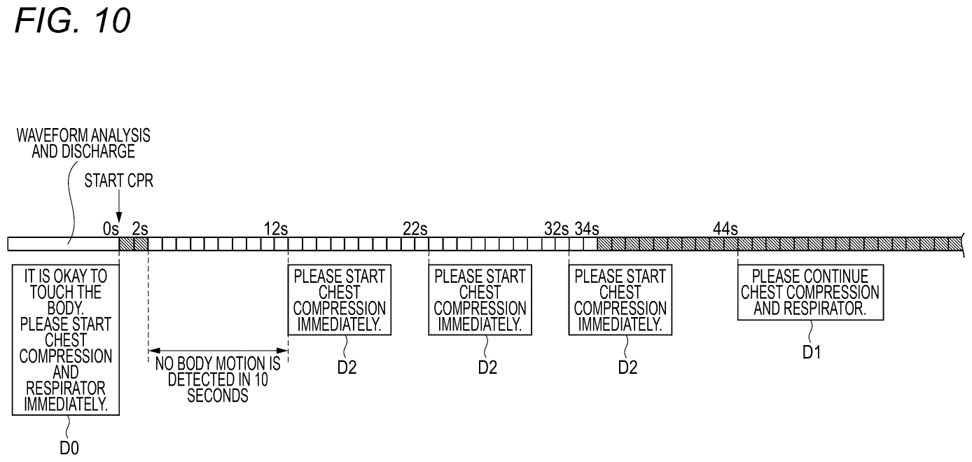

FIG. 10 is a diagram illustrating an example (a specific example 6) of outputting guidance in the CPR period by the AED 1 according to the presently disclosed subject matter. Here, it is assumed that the body motion of the subject is detected immediately after the start of the CPR period, then no body motion of the subject is detected until 34 seconds later, and the body motion of the subject is detected again after 34 seconds. In FIG. 10, a region indicating a period in which the body motion of the subject is detected is hatched.

In this case, same or similarly to the specific example 5 illustrated in FIG. 9, the pre-start guidance D0 is output before the start of the CPR period, and the first start instruction D2 is output at a timing (12 seconds after the start timing of the CPR period in the example illustrated in FIG. 10) when approximately 10 seconds have elapsed from a timing (2 seconds after the start timing of the CPR period in the example illustrated in FIG. 10) when the body motion of the subject is no longer detected. Thereafter, the start instruction D2 is output every time approximately 10 seconds elapse (in the example illustrated in FIG. 10, 22 seconds and 32 seconds after the start timing of the CPR period).

Since the body motion of the subject is detected after 35 seconds from the start timing of the CPR period, the continuation instruction D1 is output at a predetermined timing that occurs approximately every 25 seconds after 35 seconds. Here, it is assumed that 44 seconds after the start timing of the CPR period is the next output timing of the continuation instruction D1. It is assumed that the output timing of the continuation instruction D1 is not included in the output period of the start instruction D2. In such a case, the output controller 131 outputs the continuation instruction D1 at a timing 44 seconds after the start timing of the CPR period.

### (Specific Example 7) Case in Which Continuation Instruction D1 is Being Output at Output Timing of Start Instruction D2

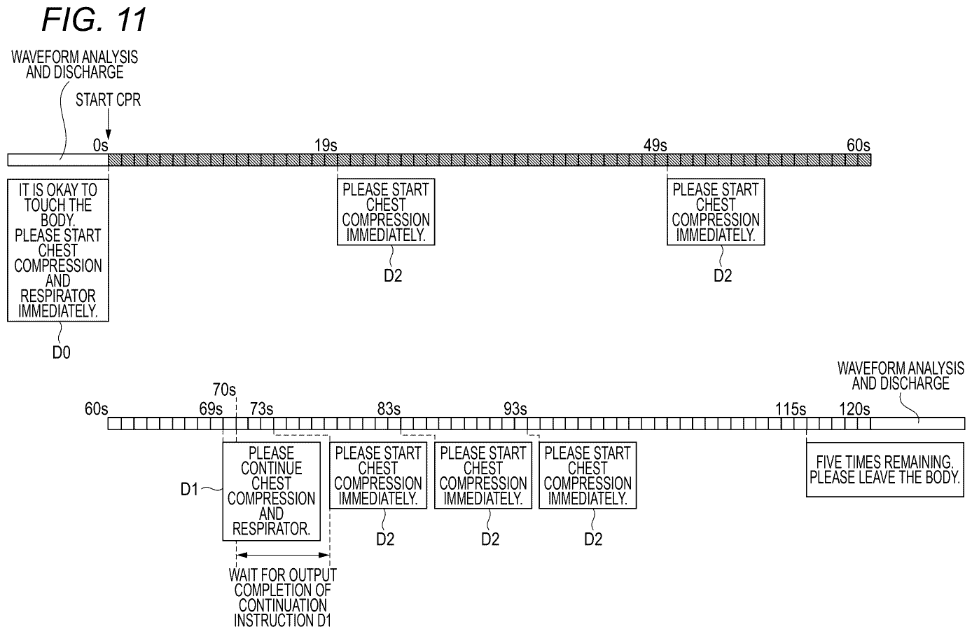

FIG. 11 is a diagram illustrating an example (a specific example 7) of outputting guidance in the CPR period by the AED 1 according to the presently disclosed subject matter. Here, it is assumed that the body motion of the subject is detected until 60 seconds after the start of the CPR period, and the body motion of the subject is not detected after 60 seconds. In FIG. 11, a region indicating a period in which the body motion of the subject is detected is hatched.

In this case, the pre-start guidance D0 is output before the start of the CPR period. The time measurement is started from the output timing of the pre-start guidance D0, and the continuation instruction D1 is output every time approximately 25 seconds elapse. In the example illustrated in FIG. 11, the continuation instruction D1 is output 18 seconds, 44 seconds, and 69 seconds after the start timing of the CPR period.

Since no body motion of the subject is detected after 60 seconds from the start timing of the CPR period, the start instruction D2 is output approximately every 10 seconds after a timing (70 seconds after the start timing of the CPR period in the example illustrated in FIG. 11) approximately 10 seconds from the timing when the body motion is no longer detected.

However, in the example illustrated in FIG. 11, the output timing (70 seconds after the start timing of the CPR period in the example illustrated in FIG. 11) of the next start instruction D2 is included in the output period of the continuation instruction D1. In such a case, the output controller 131 waits for the output of the start instruction D2 until the output of the continuation instruction D1 is completed, and outputs the start instruction D2 after the output of the continuation instruction D1 is completed (73 seconds after the start timing of the CPR period in the example illustrated in FIG. 11). Accordingly, it is possible to prevent the continuation instruction D1 and the start instruction D2 from being simultaneously output.

When the start instruction D2 is output immediately after the output of the continuation instruction D1 is completed, it may be difficult for the rescuer to understand the instruction content. Therefore, the output controller 131 may output the start instruction D2 after a predetermined time elapses after the output of the instruction of the continuation instruction D1 is completed. Accordingly, it is possible to prevent the continuation instruction D1 and the start instruction D2 from being continuously output, and to prevent the rescuer from being confused.

The output controller 131 may not output the start instruction D2 (the start instruction D2 whose output timing occurs 73 seconds after the start timing of the CPR period in the example illustrated in FIG. 11) immediately after the continuation instruction D1.

When the output timing of the start instruction D2 is included in the output period of the continuation instruction D1, the output controller 131 may interrupt the output of the continuation instruction D1 and output the start instruction D2. In such a configuration, when no body motion is detected in the last approximately 10 seconds, the start instruction D2 is preferentially output without waiting for the completion of the output of the continuation instruction D1, and thus it is possible to more clearly prompt the rescuer to resume the chest compression.

As described above, in the AED 1, the detection unit 134 detects the body motion of the subject in the CPR period after the electrocardiogram analysis and the discharge processing. In the CPR period, the output controller 131 outputs an instruction to the rescuer regarding chest compression on the subject based on the presence or absence of the body motion of the subject. The output controller 131 outputs the continuation instruction D1 instructing continuation of chest compression, and outputs the continuation instruction D1 again when the first time has elapsed from the output of the previous continuation instruction D1. When the body motion of the subject is not continuously detected for the second time shorter than the first time or longer, the output controller 131 outputs the start instruction D2 instructing the start of chest compression in preference to the continuation instruction D1, and outputs the start instruction D2 again when the second time has elapsed.

With such a configuration, when chest compression is not performed on the subject for the second time or longer, it is possible to prompt the rescuer to start chest compression, and thus it is possible to more appropriately support the movement of the rescuer in the CPR period.

Since it is possible to determine whether chest compression is performed in a cycle of the second time shorter than the first time which is an output cycle of the continuation instruction D1 of chest compression, and to output the start instruction D2 when chest compression is not performed, it is possible to shorten the interruption time when chest compression is interrupted or the like.

In the AED 1, when no body motion of the subject is detected by the output timing of the first start instruction D2 in the CPR period, the output controller 131 outputs the method notification D3 for notifying the rescuer of the method of chest compression.

With such a configuration, since the method of chest compression is notified when chest compression has not been performed in the CPR period, it is possible to grasp and perform the method of chest compression even when the rescuer does not know the method of chest compression.

In the AED 1, the output controller 131 outputs the pre-start guidance D0 regarding cardiopulmonary resuscitation of the subject before the start of the CPR period. The output controller 131 starts the time measurement for determining the output timing of the first continuation instruction D1 and the first start instruction D2 in the CPR period from the output timing of the pre-start guidance D0.

With such a configuration, the first continuation instruction D1 or the first start instruction D2 can be output earlier after the start of the CPR period. When the method notification D3 of chest compression is output at the output timing of the first start instruction D2, the method notification D3 can be output earlier.

In the AED 1, at the output timing of one instruction of the continuation instruction D1 and the start instruction D2, when the other instruction is being output, the output controller 131 outputs the one instruction after the output of the other instruction is completed.

With such a configuration, it is possible to prevent a plurality of instructions from being simultaneously output.

In the AED 1, the output controller 131 outputs the one instruction after a predetermined time elapses after the output of the other instruction is completed.

With such a configuration, it is possible to prevent a plurality of instructions from being continuously output, and to prevent the rescuer from being confused.

In the AED 1, the output controller 131 may output the continuation instruction D1 after the output of the start instruction D2 is completed when the start instruction D2 is being output at the output timing of the continuation instruction D1, and interrupt the output of the continuation instruction D1 and output the start instruction D2 when the continuation instruction D1 is being output at the output timing of the start instruction D2.

With such a configuration, the continuation instruction D1 is prevented from being output during the output of the start instruction D2, and when the chest compression on the subject is interrupted for the second time or longer, the start instruction D2 is output in preference to the continuation instruction D1. Therefore, the rescuer can be more clearly prompted to resume the chest compression.

In the AED 1, when the method notification D3 is being output at the output timing of the start instruction D2, the output controller 131 outputs the start instruction D2 after the output of the method notification D3 is completed.

With such a configuration, it is possible to prevent the start instruction D2 from being output during the output of the method notification D3. Since the rescuer hears the start instruction D2 of chest compression after hearing the method of chest compression, the rescuer can smoothly start chest compression even when the method of chest compression is unknown.

### Second Embodiment

### Configuration of AED

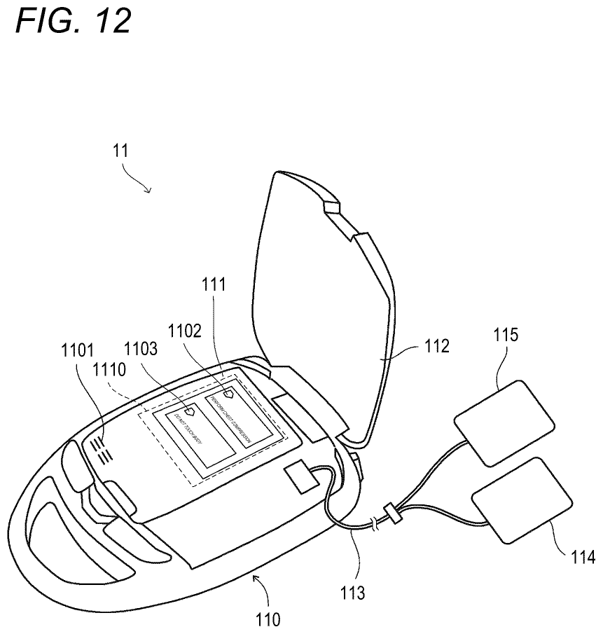

Here, the AED 11 will be described which is an example of an automated external defibrillator according to the presently disclosed subject matter. FIG. 12 is an external view of the AED 11 according to a second embodiment of the presently disclosed subject matter. As illustrated in FIG. 12, the AED 11 can include the AED body 110, the battery pack 111, the lid portion 112, the cable 113, and the pair of electrode pads 114 and 115.

The battery pack 111 is configured to supply power for operating the AED body 110 to the AED body 110. For example, the battery pack 111 is detachably connected to the back side of the AED body 110.

The lid portion 112 is a structure that covers the AED body 110. When the lid portion 112 changes from the closed state to the open state, the main power supply of the AED 11 is turned on, and the AED 11 is started up. When the lid portion 112 changes from the open state to the closed state, the main power supply of the AED 11 is turned off. The AED 11 is not limited to a configuration in which the main power supply is switched ON/OFF in response to opening and closing of the lid portion 112. For example, a power button configured to switch the main power supply ON/OFF may be provided on the AED body 110.

Each of the electrode pads 114 and 115 can include a gel having viscosity. When the AED 11 is not in use, the electrode pads 114 and 115 are accommodated in a bag, in a state in which release paper is attached to each gel. The bag that accommodates the electrode pads 114 and 115 is attached to the inside of the lid portion 112, for example. A rescuer who uses the AED 11 removes the bag from the lid portion 112 and takes out the electrode pads 114 and 115 from the bag. Then, the rescuer attaches the gel portion to the skin of a subject in a state in which the electrode pads 114 and 115 are peeled off from the release paper.

The cable 113 electrically connects the AED body 110 and the electrode pads 114 and 115. In a state in which the electrode pads 114 and 115 are attached to the subject, the AED body 110 performs processing such as electrocardiogram analysis and electric shock (hereinafter also referred to as “discharge processing”) on the subject.

### Configuration of AED Body

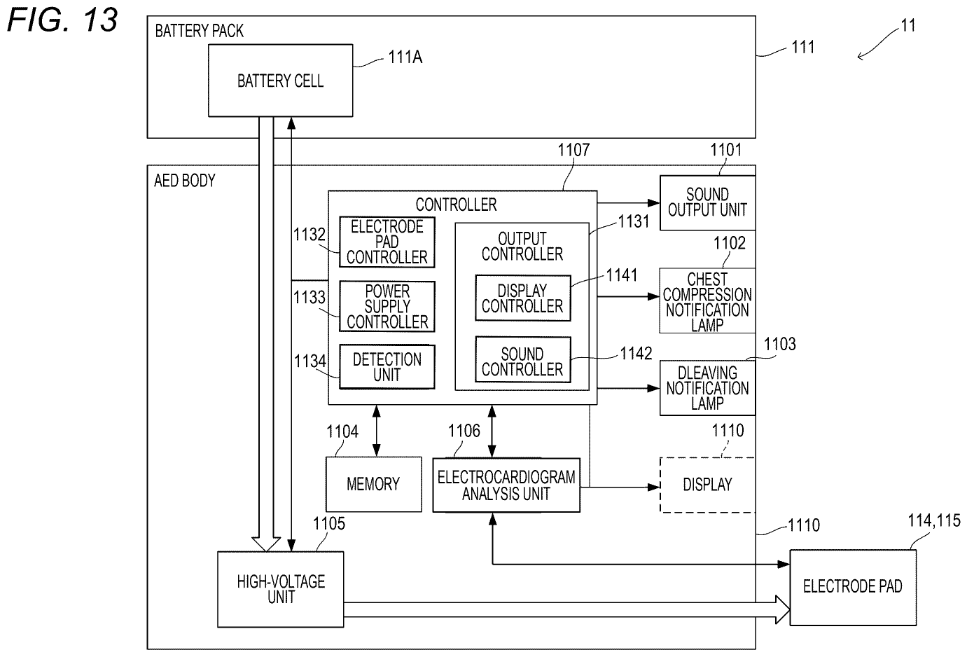

Next, the configuration of the AED body 110 will be described. FIG. 13 is a block diagram illustrating the configuration of the AED 11 illustrated in FIG. 12. As illustrated in FIGS. 12 and 13, the AED body 110 can include the sound output unit 1101, a chest compression notification lamp (an example of a display unit) 1102, and a leaving notification lamp (an example of a display unit) 1103. As illustrated in FIG. 13, the AED body 110 can further include the memory 1104, the high-voltage unit 1105, the electrocardiogram analysis unit 1106, and a controller 1107. The memory 1104 is configured to store a program or the like for controlling various operations of the AED 11.

When the AED 11 is started up, the controller 1107 reads and executes the program or the like that is stored in the memory 1104, and controls the various operations of the AED 11. The controller 1107 can include a central processing unit (CPU), a read only memory (ROM), a random access memory (RAM), a hard disk drive (HDD), and the like. More specifically, the controller 1107 is configured to function as the output controller 1131, the electrode pad controller 1132, the power supply controller 1133, and the detection unit 1134. The output controller 1131 can include a display controller 1141 and a sound controller 1142.

The display controller 1141 is configured to control lighting or blinking of the chest compression notification lamp 1102 and the leaving notification lamp 1103.

The sound controller 1142 is configured to read sound data such as voice guidance and warning sound for the rescuer from the memory 1104, and to output the read sound data to the sound output unit 1101. The sound output unit 1101 is, for example, a speaker. Upon receiving the sound data output from the sound controller 1142, the sound output unit 1101 outputs voice guidance or warning sound based on the sound data.

The electrode pad controller 1132 is configured to control the electrode pads 114 and 115 and the electrocardiogram analysis unit 1106 such that the electrocardiogram analysis unit 1106 executes the electrocardiogram analysis. That is, the electrocardiogram analysis unit 1106 is configured to execute electrocardiogram analysis of the subject through the electrode pads 114 and 115 by being controlled by the electrode pad controller 1132.

After the electrocardiogram analysis by the electrode pad controller 1132, the power supply controller 1133 executes control such that discharge processing on the subject is performed. Specifically, the power supply controller 1133 is configured to control the battery cell 111A provided in the battery pack 111 and the internal capacitor of the high-voltage unit 1105 such that the internal capacitor is charged by the battery cell 111A and discharged from the electrode pads 114 and 115.

The detection unit 1134 is configured to detect whether the rescuer is in contact with the subject. For example, the detection unit 1134 is configured to obtain the impedance between the pair of electrode pads 114 and 115, and detect whether the rescuer is in contact with the subject based on a change in the impedance.

### Details of Notification by Output Controller

### Outline of Operations

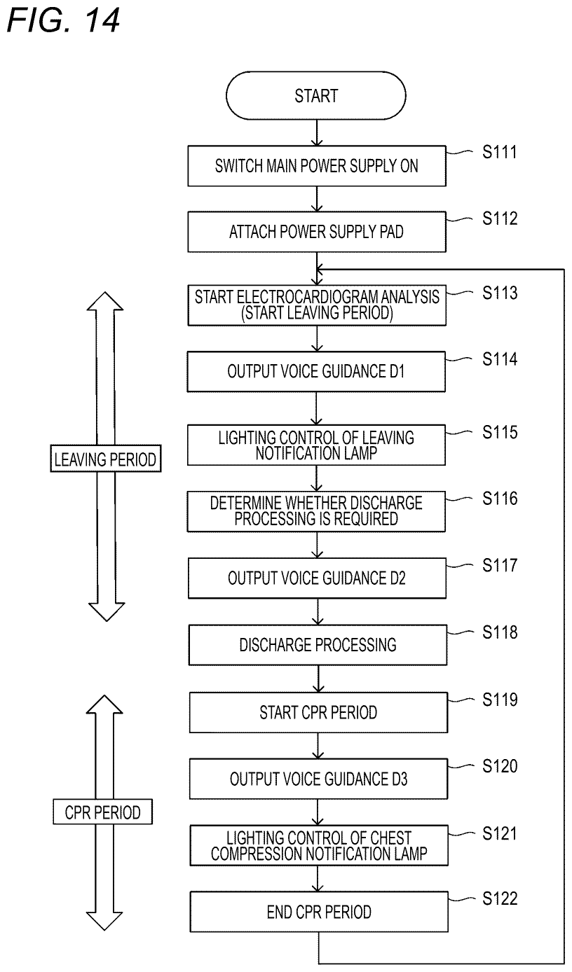

FIG. 14 is a flowchart illustrating an outline of a procedure of operations of the AED 11 illustrated in FIG. 13. Referring to FIGS. 13 and 14, first, it is assumed that the main power supply of the AED 11 is switched on (step S111) and the electrode pads 114 and 115 are attached to the subject (step S112). In this case, the electrode pad controller 1132 controls the electrocardiogram analysis unit 1106 to execute the electrocardiogram analysis, and the electrocardiogram analysis is started. That is, the leaving period is started (step S113).

When the leaving period is started, the sound controller 1142 causes the sound output unit 1101 to output voice guidance D1 that “Please leave the body”, for example (step S114).

Then, the display controller 1141 controls blinking or lighting of the leaving notification lamp 1103 (hereinafter, simply referred to as “lighting control”) in order to visually notify the rescuer of the leaving from the subject, in the leaving period from the start of the electrocardiogram analysis to the start of the discharge processing to be described later (step S115). At this time, the display controller 1141 changes, for example, the blinking cycle of the leaving notification lamp 1103, according to the detection result obtained by the detection unit 1134, which is whether the rescuer is in contact with the subject. Such lighting control of the leaving notification lamp 1103 (step S115) is continuously executed from the start to the end of the leaving period, which is during a period from the start of the electrocardiogram analysis (step S113) to the start of the discharge processing (step S118) to be described later. Details of the lighting control of the leaving notification lamp 1103 during the leaving period will be described later.

Next, when the electrocardiogram analysis ends, the power supply controller 1133 determines whether the discharge processing on the subject is necessary, based on the result of the electrocardiogram analysis (step S116). Here, it is assumed that the power supply controller 1133 determines that the discharge processing is necessary. In this case, the display controller 1141 causes the sound output unit 1101 to output, for example, voice guidance D2 that “Electric shock is to be performed” (step S117).

Next, the electrode pad controller 1132 executes control such that the discharge processing on the subject is performed (step S118). Then, when the discharge processing on the subject is performed, the CPR period is started (step S119). When it is determined in step S116 that the discharge processing is unnecessary, the CPR period is started without performing the discharge processing (step S118). When the CPR period is started, the sound controller 1142 in the output controller 1131 causes the sound output unit 1101 to output, for example, voice guidance D3 that “Please start chest compression and respirator” (step S120).

Then, the display controller 1141 in the output controller 1131 controls blinking or lighting of the chest compression notification lamp 1102 (hereinafter, simply referred to as “lighting control”), in order to visually notify the rescuer of the execution of chest compression on the subject in the CPR period (step S121). At this time, the display controller 1141 changes, for example, the blinking cycle of the chest compression notification lamp 1102 according to the detection result obtained by the detection unit 1134, which is whether the rescuer is in contact with the subject. Such lighting control of the chest compression notification lamp 1102 (step S121) is continuously executed from the start of the CPR period (step S119) to the end of the CPR period (step S122) to be described later. Details of the lighting control of the chest compression notification lamp 1102 in the CPR period will be described later.

When a predetermined time elapses from the start timing of the CPR period, the CPR period ends (step S122). Then, the leaving period is started again, and the operation after S113 is repeated.

### Lighting Control of Leaving Notification Lamp in Leaving Period

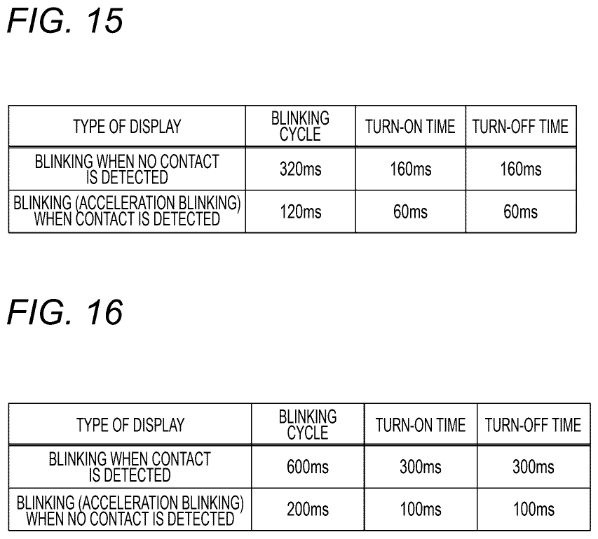

FIG. 15 is a table illustrating a specific example of lighting control (step S115 illustrated in FIG. 14) of the leaving notification lamp 1103 in the leaving period by the display controller 1141 illustrated in FIG. 13. Referring to FIGS. 13 and 15, in the leaving period, the display controller 1141 executes control such that the blinking cycle of the leaving notification lamp 1103 in a case where the contact with the subject is detected is shorter than in a case where no contact with the subject is detected, for example.

Specifically, when no contact with the subject is detected in the leaving period, the leaving notification lamp 1103 is controlled such that, for example, each of the turn-on time and the turn-off time is 160 ms and the blinking cycle is 320 ms. On the other hand, when the contact with the subject is detected in the leaving period, the leaving notification lamp 1103 is controlled such that, for example, each of the turn-on time and the turn-off time is 60 ms and the blinking cycle is 120 ms. Hereinafter, such control for shortening the blinking cycle is referred to as “acceleration blinking control”.

In this way, in the leaving period, when the contact with the subject is detected, the blinking cycle of the leaving notification lamp 1103 is shortened to approximately ⅓ as compared with the case in which no contact with the subject is detected. Accordingly, it is possible to make the leaving notification lamp 1103 conspicuous and make it easy to visually notice the leaving notification lamp 1103, and thus it is possible to more effectively prompt the rescuer to leave the subject.

The display controller 1141 is not limited to controlling the blinking cycle of the leaving notification lamp 1103 to be different between the case in which the contact with the subject is detected and the case in which no contact with the subject is detected. For example, the display controller 1141 may change the lighting color of the leaving notification lamp 1103 between the case in which the contact with the subject is detected and the case in which no contact with the subject is detected.

### Lighting Control of Chest Compression Notification Lamp in CPR Period

FIG. 16 is a table illustrating a specific example of lighting control (step S121 illustrated in FIG. 14) of the chest compression notification lamp 1102 in the CPR period by the display controller 1141 illustrated in FIG. 13. Referring to FIGS. 13 and 16, in the CPR period, when no contact of the rescuer with the subject is detected by the detection unit 1134, chest compression may not be performed on the subject. Therefore, in the CPR period, the display controller 1141 executes control such that the blinking cycle of the chest compression notification lamp 1102 is shorter when no contact with the subject is detected than when the contact with the subject is detected, for example.

Specifically, when the contact with the subject is detected in the CPR period, the chest compression notification lamp 1102 is controlled such that, for example, each of the turn-on time and the turn-off time is 300 ms and the blinking cycle is 600 ms.

The blinking cycle of the chest compression notification lamp 1102 when the contact with the subject is detected in the CPR period is set based on the cycle of chest compression or the like defined in JRC Resuscitation Guidelines 2020″. That is, the blinking of the chest compression notification lamp 1102 plays a role of making a notification of the timing of performing chest compression. The turn-on time, turn-off time, and blinking cycle of the chest compression notification lamp 1102 when the contact with the subject is detected in the CPR period can be set by the user.

On the other hand, it is assumed that no contact with the subject is detected in the CPR period. In this case, the chest compression notification lamp 1102 is subjected to the acceleration blinking control such that, for example, each of the turn-on time and the turn-off time is 100 ms and the blinking cycle is 200 ms.

In this way, in the CPR period, when no contact with the subject is detected, the blinking cycle of the chest compression notification lamp 1102 is shortened to approximately ⅓ as compared with the case in which the contact with the subject is detected. Accordingly, it is possible to make the chest compression notification lamp 1102 conspicuous and make it easy to visually notice the chest compression notification lamp 1102, and thus it is possible to more effectively prompt the rescuer to perform chest compression on the subject.

Here, the instruction for leaving to the rescuer in the leaving period is more urgent than the instruction for chest compression to the rescuer in the CPR period, and is highly required to be conspicuous. Therefore, the blinking cycle (120 ms in the example illustrated in FIG. 15) when the acceleration blinking control of the leaving notification lamp 1103 is executed is set to be shorter than the blinking cycle (200 ms in the example illustrated in FIG. 16) when the acceleration blinking control of the chest compression notification lamp 1102 is executed.

The display controller 1141 is not limited to controlling the blinking cycle of the chest compression notification lamp 1102 to be different between the case in which the contact with the subject is detected and the case in which no contact with the subject is detected. For example, the display controller 1141 may change the lighting color of the chest compression notification lamp 1102 between the case in which the contact with the subject is detected and the case in which no contact with the subject is detected.

### Notification by Display

Referring again to FIG. 12, the AED body 110 may be provided with a display (an example of a display unit) 1110 at a position indicated by a broken line or the like. In this case, the display controller 1141 illustrated in FIG. 13 causes the display 1110 to display characters such as “please leave the body” in the leaving period. The display controller 1141 causes the display 1110 to display characters such as “please start chest compression and respirator” in the CPR period.

In the leaving period, it is assumed that the body motion of the subject is detected by the detection unit 1134. In this case, the display controller 1141 can change the display mode of the display 1110 as compared with the case in which no body motion of the subject is detected. For example, in addition to displaying characters, the display controller 1141 executes at least one of changing the background color of the display 1110, changing the background pattern of the display 1110, and changing the color of characters, or blinks the background or characters of the display 1110.

Accordingly, when the rescuer is in contact with the subject in the leaving period, it is possible to make the display 1110 conspicuous and make it easy to visually notice the display 1110, and thus it is possible to more effectively prompt the rescuer to leave the subject.

It is assumed that no body motion of the subject is detected by the detection unit 1134 in the CPR period. In this case, the display controller 1141 can change the display mode of the display 1110 as compared with the case in which the body motion of the subject is detected. For example, in addition to displaying characters, the display controller 1141 executes at least one of changing the background color of the display 1110, changing the background pattern of the display 1110, and changing the color of characters, or blinks the background or characters of the display 1110.

Accordingly, when the rescuer is not in contact with the subject in the CPR period, that is, when the chest compression is not performed, it is possible to make the display 1110 conspicuous and make it easy to visually notice the display 1110, and thus it is possible to more effectively prompt the rescuer to perform the chest compression on the subject.

As described above, in the AED 11 according to the second embodiment of the presently disclosed subject matter, the detection unit 1134 detects whether the rescuer is in contact with the subject. The display controller 1141 changes the blinking cycle or the lighting color of the display units such as the chest compression notification lamp 1102, the leaving notification lamp 1103, and the display 1110 provided in the AED 1 according to the detection result obtained by the detection unit 1134.

In this way, by the configuration in which the blinking cycle or the lighting color of the display unit is changed according to whether the rescuer is in contact with the subject, it is possible to more effectively instruct the rescuer.

In the AED 11, the display controller 1141 executes control to blink or turn on the display units such as the leaving notification lamp 1103 and the display 1110 during the leaving period from the start of the electrocardiogram analysis on the subject to the start of the discharge processing. The display controller 1141 controls the blinking cycle or the lighting color of the display unit to be different between the case in which the contact with the subject is detected and the case in which no contact with the subject is detected in the leaving period.

With such a configuration, when the rescuer is in contact with the subject in the leaving period, the blinking cycle or the lighting color of the display units such as the leaving notification lamp 1103 and the display 1110 is changed, and thus it is possible to more effectively prompt the rescuer to leave the subject.

In the AED 11, the display controller 1141 executes control to blink the leaving notification lamp 1103 during the leaving period. In the leaving period, the display controller 1141 executes control such that the blinking cycle of the leaving notification lamp 1103 is shorter when the contact with the subject is detected than in the leaving period when no contact with the subject is detected.

In this way, when the rescuer is in contact with the subject in the leaving period, the rescuer can easily notice that the rescuer is to leave the subject by shortening the blinking cycle of the leaving notification lamp 1103 and making the leaving notification lamp 1103 conspicuous.

In the AED 11, the display controller 1141 executes control to blink or turn on the display units such as the chest compression notification lamp 1102 and the display 1110 in the CPR period after the discharge processing is performed on the subject. The display controller 1141 executes control such that the blinking cycle or the lighting color of the display unit is different between the case in which no contact with the subject is detected and the case in which the contact with the subject is detected in the CPR period.

With such a configuration, when the rescuer is not in contact with the subject in the CPR period, that is, when chest compression is not performed, the blinking cycle or the lighting color of the display units such as the chest compression notification lamp 1102 and the display 1110 is changed, and thus it is possible to more effectively prompt the rescuer to perform the chest compression.

In the AED 1, the display controller 1141 executes control to blink the chest compression notification lamp 1102 in the CPR period. In the CPR period, the display controller 1141 executes control such that the blinking cycle of the chest compression notification lamp 1102 is shorter when no contact with the subject is detected than when the contact with the subject is detected.

In this way, when the rescuer is not in contact with the subject in the CPR period, that is, when the chest compression is not performed, the rescuer can easily notice that the chest compression is to be performed by shortening the blinking cycle of the chest compression notification lamp 1102 and making the chest compression notification lamp 1102 conspicuous.

The foregoing description of the exemplary embodiments of the present invention has been provided for the purposes of illustration and description. It is not intended to be exhaustive or to limit the invention to the precise forms disclosed. Obviously, many modifications and variations will be apparent to practitioners skilled in the art. The embodiments were chosen and described in order to best explain the principles of the invention and its practical applications, thereby enabling others skilled in the art to understand the invention for various embodiments and with the various modifications as are suited to the particular use contemplated. It is intended that the scope of the invention be defined by the following claims and their equivalents.

What is claimed is:

1. An automated external defibrillator for performing electrocardiogram analysis and discharge processing on a subject, the automated external defibrillator comprising:
a detection unit configured to detect a body motion of the subject, in a cardiopulmonary resuscitation period after the electrocardiogram analysis and the discharge processing; and
an output controller configured to output, to a rescuer, an instruction regarding chest compression on the subject, based on presence or absence of the body motion of the subject, in the cardiopulmonary resuscitation period,
wherein the output controller is configured to:
output a continuation instruction for instructing continuation of chest compression;
in a case where a first time has elapsed from the output of a previous continuation instruction, output the continuation instruction again;
in a case where no body motion of the subject is continuously detected for a second time shorter than the first time or for longer than the second time, output a start instruction for instructing a start of the chest compression in priority to the continuation instruction; and
in a case where the second time has elapsed from the output of a previous start instruction, output the start instruction again.

2. The automated external defibrillator according to claim 1,
wherein the output controller is configured to, in a case where no body motion of the subject is detected until an output timing of a first start instruction in the cardiopulmonary resuscitation period, output a method notification for transmitting a method of chest compression to the rescuer.

3. The automated external defibrillator according to claim 1,
wherein the output controller is configured to:
output guidance related to cardiopulmonary resuscitation of the subject, before a start of the cardiopulmonary resuscitation period; and
start time measurement, from an output timing of the guidance, for determining output timing of a first continuation instruction and a first start instruction in the cardiopulmonary resuscitation period.

4. The automated external defibrillator according to claim 1,
wherein, in a case where one instruction of the continuation instruction and the start instruction is being output at an output timing of another instruction of the continuation instruction and the start instruction, the output controller is configured to output the other instruction after the output of the one instruction is completed.

5. The automated external defibrillator according to claim 4,
wherein the output controller is configured to output the other instruction after a predetermined time elapses after the output of the one instruction is completed.

6. The automated external defibrillator according to claim 1,
wherein the output controller is configured to:
in a case where the start instruction is being output at an output timing of the continuation instruction, output the continuation instruction after the output of the start instruction is completed, and
in a case where the continuation instruction is being output at an output timing of the start instruction, interrupt the output of the continuation instruction and output the start instruction.

7. The automated external defibrillator according to claim 2,
wherein the output controller is configured to, in a case where the method notification is being output at an output timing of the start instruction, output the start instruction after the output of the method notification is completed.

8. An automated external defibrillator for performing electrocardiogram analysis and discharge processing on a subject, the automated external defibrillator comprising:
a detection unit configured to detect whether a rescuer is in contact with the subject; and
a display controller configured to change a blinking cycle or a lighting color of a display unit provided in the automated external defibrillator, according to a detection result obtained by the detection unit.

9. The automated external defibrillator according to claim 8,
wherein the display controller is configured to:
execute control to blink or turn on the display unit during a leaving period from a start of the electrocardiogram analysis on the subject to a start of the discharge processing; and
execute control such that the blinking cycle or the lighting color of the display unit is different between a case in which the contact is detected and a case in which the contact is not detected, in the leaving period.

10. The automated external defibrillator according to claim 9,
wherein the display unit is a lamp provided in the automated external defibrillator, and
wherein the display controller is configured to:
execute control to blink the lamp during the leaving period; and
execute control such that the blinking cycle of the lamp in a case where the contact is detected is shorter than in a case where the contact is not detected, during the leaving period.

11. The automated external defibrillator according to claim 8,
wherein the display controller is configured to:
execute control to blink or turn on the display unit in a cardiopulmonary resuscitation period after the discharge processing is performed on the subject; and
execute control such that the blinking cycle or the lighting color of the display unit is different between a case in which the contact is not detected and a case in which the contact is detected, in the cardiopulmonary resuscitation period.

12. The automated external defibrillator according to claim 11,
wherein the display unit is a lamp provided in the automated external defibrillator, and
wherein the display controller is configured to:
execute control to blink the lamp during the cardiopulmonary resuscitation period; and
execute control such that the blinking cycle of the lamp in a case where the contact is not detected is shorter than in a case where the contact is detected, in the cardiopulmonary resuscitation period.
